(19) United States
(12) Patent Application Publication
(10) Pub. No.: US 2018/0214663 A1
(43) Pub. Date: Aug. 2, 2018

(54) LIGHTING SYSTEM AND INFORMATION PRESENTATION METHOD

(71) Applicant: Panasonic Intellectual Property Management Co., Ltd., Osaka (JP)

(72) Inventors: Saori UENO, Osaka (JP); Kazuhiro HATTA, Osaka (JP)

(21) Appl. No.: 15/879,940

(22) Filed: Jan. 25, 2018

(30) Foreign Application Priority Data
Jan. 31, 2017 (JP) 2017-015135

(51) Int. Cl. A61M 21/02; G06F 3/14

(52) U.S. Cl. CPC A61M 21/02; A61N 2005/0627; G06F 3/14

(57) ABSTRACT

A lighting system includes a light source unit; an irradiation controller that forms a first area in a space by causing the light source unit to emit first light according to a first irradiation schedule, and simultaneously with the formation of the first area, forms a second area in the space by causing the light source unit to emit second light according to a second irradiation schedule; a communicator that acquires health condition information indicating the health condition of a user; a judgment unit that judges whether the user is a first subject targeted for irradiation with the first light or a second subject targeted for irradiation with the second light, on the basis of the acquired health condition information; and a presentation unit that presents whether the user is the first subject or the second subject, on the basis of the judgment.

### CROSS REFERENCE TO RELATED APPLICATION

This application claims the benefit of priority of Japanese Patent Application Number 2017-015135 filed on Jan. 31, 2017, the entire content of which is hereby incorporated by reference.

### BACKGROUND

### 1. Technical Field

The present disclosure relates to a lighting system for use in phototherapy.

### 2. Description of the Related Art

Phototherapy for improving the health conditions of users by photoirradiation has conventionally been proposed. Japanese Unexamined Patent Application Publication No. 2015-2832 discloses a sleeping room in which the luminous environment during sleep is approximated to the natural sunlight cycle so that a user can get a high quality sleep.

### SUMMARY

When phototherapy is carried out in facilities such as welfare facilities for the aged, it is necessary to adjust photoirradiation schedules (e.g., timing of photoirradiation and illuminance) according to the health conditions of users in order to improve the effectiveness of the phototherapy, because the users in such facilities are in various health conditions. However, since phototherapy is not yet common, it is difficult for the staff of welfare facilities for the aged who has no expert knowledge about phototherapy to adjust the photoirradiation schedules according to the health conditions of users.

One aspect of the present disclosure provides a lighting system capable of offering phototherapy according to the health conditions of users.

A lighting system according to an aspect of the present disclosure includes a light source unit, an irradiation controller, an acquisition unit, a judgment unit, and a presentation unit. The irradiation controller forms a first area that is irradiated with first light in a space by causing the light source unit to emit the first light according to a first irradiation schedule, and simultaneously with formation of the first area, forms a second area that is irradiated with second light in the space by causing the light source unit to emit the second light according to a second irradiation schedule different from the first irradiation schedule. The acquisition unit acquires health condition information that indicates a health condition of a user. The judgment unit performs judgment as to whether the user is a first subject targeted for irradiation with the first light or a second subject targeted for irradiation with the second light, in accordance with the health condition information acquired. The presentation unit presents a result of the judgment.

The lighting system according to this aspect of the present disclosure is capable of offering phototherapy according to the health conditions of users.

### BRIEF DESCRIPTION OF DRAWINGS

The figures depict one or more implementations in accordance with the present teaching, by way of examples only, not by way of limitations. In the figures, like reference numerals refer to the same or similar elements.

FIG. 1 illustrates an overview of a lighting system according to Embodiment 1;

FIG. 2 is a block diagram illustrating a functional configuration of the lighting system according to Embodiment 1;

FIG. 3 illustrates an example of an image displayed by a presentation unit according to Embodiment 1;

FIG. 4 is a sequence diagram illustrating an example of operations of the lighting system according to Embodiment 1.

FIG. 5 illustrates sleeping hours of a first subject who does not have an advanced sleep phase pattern;

FIG. 6 illustrates sleeping hours of a second subject who has an advanced sleep phase pattern;

FIG. 7 illustrates an example of a first irradiation schedule that is used when health condition information indicates the sleep state of a user;

FIG. 8 illustrates an example of a second irradiation schedule that is used when the health condition information indicates the sleep state of a user;

FIG. 9 illustrates an example of an emission spectrum of light with a first color temperature;

FIG. 10 illustrates an example of an emission spectrum of light with a second color temperature;

FIG. 11 illustrates an example of the first irradiation schedule that is used when the health condition information indicates the degree of dementia of a user;

FIG. 12 illustrates an example of the second irradiation schedule that is used when the health condition information indicates the degree of dementia of a user;

FIG. 13 is a diagram for illustrating how an irradiation controller changes the sizes of first and second areas;

FIG. 14 illustrates an irradiation schedule for users who are judged to have delayed sleep phases and severe dementia;

FIG. 15 illustrates an irradiation schedule for users who are judged to have advanced sleep phases and mild dementia;

FIG. 16 illustrates a variation of a presentation given by a presentation device;

FIG. 17 illustrates an overview of a lighting system according to Embodiment 2;

FIG. 18 is a block diagram illustrating a functional configuration of the lighting system according to Embodiment 2; and

FIG. 19 illustrates an example of an image displayed by the presentation unit according to Embodiment 1.

### DETAILED DESCRIPTION OF THE EMBODIMENTS

Hereinafter, embodiments will be described with reference to the drawings. Each embodiment described below illustrates one generic or specific example of the present disclosure. Thus, numerical values, shapes, materials, constituent elements, arrangement positions and forms of connection of the constituent elements, steps, sequence of steps, and so on in the following embodiments are mere examples and do not intend to limit the scope of the present disclosure. Among the constituent elements described in the following embodiments, those that are not recited in any independent claim, which represents the broadest concept, are described as optional constituent elements.

Note that each drawing is a schematic diagram and does not necessarily provide precise depiction. The substantially same structural elements are given the same reference signs throughout the drawings, and their detailed description may be omitted or simplified.

### Embodiment 1

Overview of Lighting System

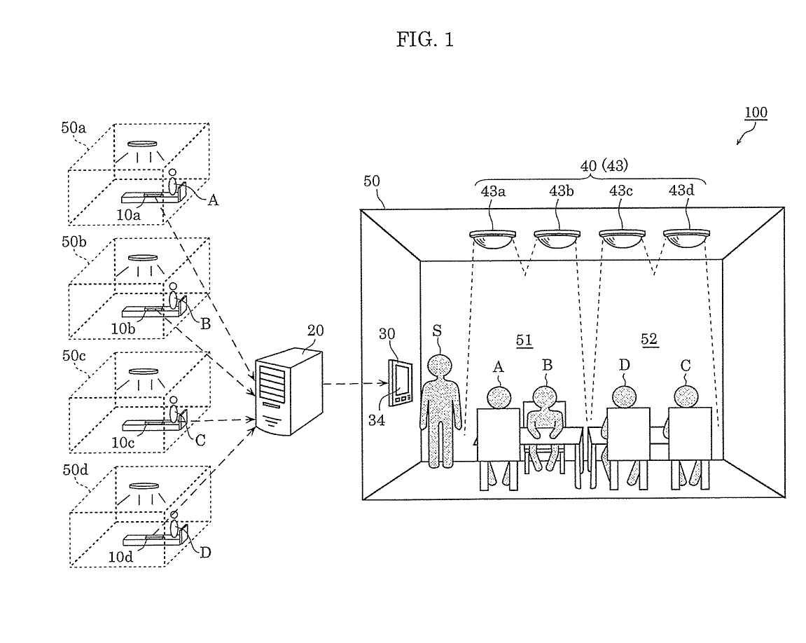

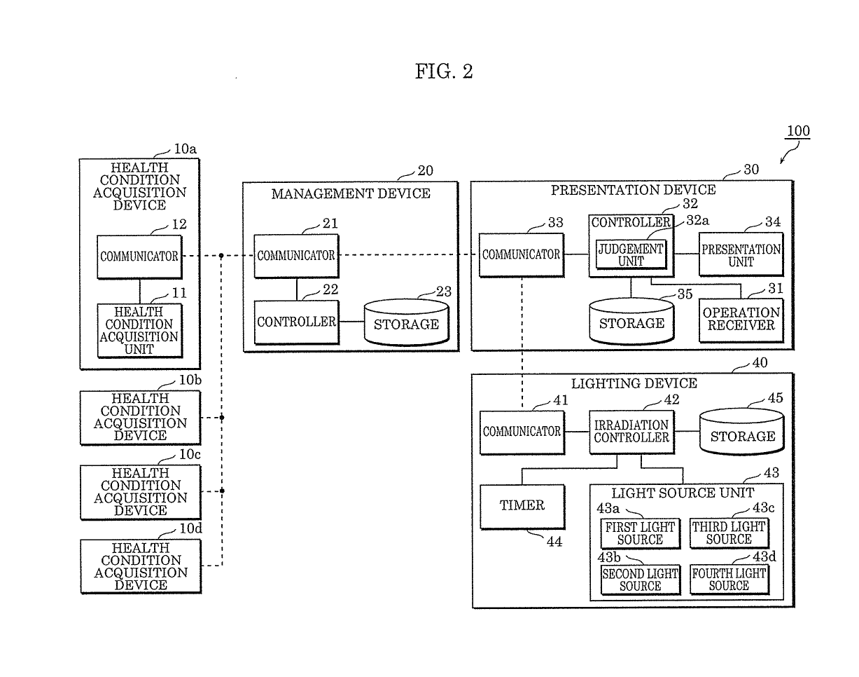

First, an overall configuration of an information presentation system according to Embodiment 1 will be described. FIG. 1 illustrates an overview of a lighting system according to Embodiment 1. FIG. 2 is a block diagram illustrating a functional configuration of the lighting system according to Embodiment 1.

Lighting system 100 illustrated in FIGS. 1 and 2 is a system for offering phototherapy in space 50 such as a lounge or a dining room where a plurality of users can stay at the same time in facilities such as welfare facilities for the aged. Lighting system 100 includes health condition acquisition devices 10a to 10d, management device 20, presentation device 30, and lighting device 40.

In lighting system 100, health condition acquisition devices 10a to 10d may respectively be disposed in private rooms 50a to 50d for users A to D and acquire health condition information that indicates the health conditions of users A to D. The acquired health condition information for users A to D is collected by management device 20 and stored in storage 23 of management device 20.

Phototherapy may be started by staff S of the facility operating presentation device 30. When staff S has operated presentation device 30, lighting device 40 emits light according to predetermined irradiation schedules for use in phototherapy. At this time, lighting device 40 forms at least two types of irradiation areas in order to irradiate users A to D with light according to their health conditions.

Specifically, some light sources of lighting device 40 form first area 51 that is irradiated with first light in space 50 by emitting the first light according to a first irradiation schedule. Simultaneously with the formation of first area 51, the other light sources of lighting device 40 form second area 52 that is irradiated with second light in space 50 by emitting the second light according to a second irradiation schedule. That is, space 50 is irradiated with the first light and the second light simultaneously (at the same time), so that first area 51 and second area 52 are formed simultaneously (at the same time). While the example shows a case where first area 51 is formed in a different place from second area 52, first and second areas 51 and 52 may overlap each other.

Presentation device 30 judges whether each of users A to D is a first subject targeted for irradiation with the first light or a second subject targeted for irradiation with the second light, on the basis of the health condition information stored in storage 23 of management device 20, and presents (displays) the judgment results to (on) presentation unit (display) 34.

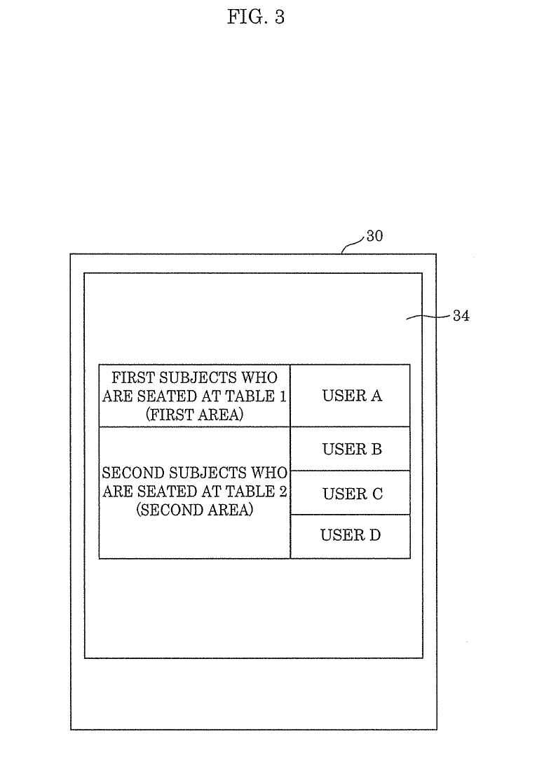

Specifically, presentation unit 34 displays an image as illustrated in FIG. 3. FIG. 3 illustrates an example of the image displayed on presentation unit 34. This image enables staff S to guide user A to have a seat at a first table arranged in first area 51 and to guide users B, C, and D to have seats at a second table arranged in second area 52.

In this way, even if staff S has no expert knowledge about phototherapy, lighting system 100 enables the users to receive phototherapy according to their health conditions. A detailed configuration of each device in lighting system 100 will be described hereinafter with reference to FIGS. 1 and 2.

Health Condition Acquisition Device

First, health condition acquisition devices 10a to 10d will be described. While the following description takes health condition acquisition device 10a as an example, health condition acquisition devices 10b to 10d also have similar configurations.

Health condition acquisition device 10a may be a device that is disposed in private room 50a for user A in the facility and acquires the health condition information indicating the health condition of user A. Health condition acquisition device 10a includes health condition acquisition unit 11 and communicator 12.

Health condition acquisition unit 11 acquires the health condition information indicating the health condition of user A. The health condition information may be information that indicates the sleep state of user A, information that indicates the degree of dementia of user A, or information that indicates the activity level of user A, but there are no particular limitations on the health condition information. When the health condition information indicates the sleep state of user A, health condition acquisition unit 11 may be a sleep measuring meter that includes a pad-type (sheet-type) sensor and detects whether user A is present on the sensor. Health condition acquisition unit 11 detects a period of time during which user A is present on the sensor, i.e., a period of time during which user A is sleeping. In other words, health condition acquisition unit 11 acquires the period of time during which user A is sleeping as information (health condition information) indicating the sleep state. The acquired health condition information (detected sleeping hours) is transmitted to management device 20 by communicator 12.

Communicator 12 transmits the health condition information to management device 20. Specifically, communicator 12 is a communication module (communication circuit) that allows health condition acquisition device 10a to communicate with management device 20. The communication between health condition acquisition device 10a and management device 20 may be wired communication, or may be wireless communication. There are no particular limitations on communication standards for use in this communication.

Management Device

Next, management device 20 will be described. Management device 20 receives the health condition information about users A to D from health condition acquisition devices 10a to 10d and accumulates (stores) the received health condition information. Management device 20 also transmits the health condition information to presentation device 30 upon receipt of a request for the health condition information from presentation device 30.

Management device 20 may be a server installed in the aforementioned facility, or may be a cloud server installed outside the facility. Specifically, management device 20 includes communicator 21, controller 22, and storage 23.

Communicator 21 receives the health condition information about users A to D from health condition acquisition devices 10a to 10d. Communicator 21 also transmits the health condition information stored in storage 23 to presentation device 30 under the control of controller 22. Specifically, communicator 21 is a communication module (communication circuit) that allows management device 20 to communicate with health condition acquisition devices 10a to 10d and presentation device 30.

The communication between management device 20 and health condition acquisition devices 10a to 10d may be wired communication, or may be wireless communication. There are no particular limitations on communication standards for use in this communication. The communication between management device 20 and presentation device 30 may be wired communication, or may be wireless communication. There are no particular limitations on communication standards for use in this communication. When the communication standards used between management device 20 and health condition acquisition devices 10a to 10d differ from the communication standards used between management device 20 and presentation device 30, communicator 21 may be configured by a plurality of types of communication modules (communication circuits).

Controller 22 performs information processing such as storing the health condition information in storage 23. When communicator 21 has received a request for the health condition information, controller 22 reads out health condition information corresponding to the request from storage 23 and causes communicator 21 to transmit the read health condition information. Specifically, controller 22 is implemented by, for example, a processor, a microcomputer, or a dedicated circuit.

Storage 23 is a storage device that stores the health condition information received by communicator 21. The health condition information is stored with an identifier different for each user in a manner that enables the health condition information for each of users A to D to be distinguishable. Storage 23 also stores other information such as control programs executed by controller 22. Specifically, storage 23 is implemented by, for example, a hard disc drive (HDD) or a semiconductor memory.

Presentation Device

Next, presentation device 30 will be described. Presentation device 30 presents whether each of users A to D is the first subject targeted for irradiation with the first light or the second subject targeted for irradiation with the second light, on the basis of the health condition information stored in management device 20. That is, presentation device 30 presents to staff S whether each of users A to D is the first subject who is seated at the first table arranged in first area 51 or the second subject who is seated at the second table arranged in second area 52. Presentation device 30 also functions as a controller of lighting device 40.

Presentation device 30 may be a device dedicated to lighting system 100 and installed in space 50. Alternatively, presentation device 30 may be installed in a place other than space 50. Presentation device 30 may be a general-purpose device such as a smartphone or a tablet terminal. Presentation device 30 includes operation receiver 31, controller 32, communicator 33, presentation unit 34, and storage 35.

Operation receiver 31 receives a predetermined operation from staff S to start phototherapy. Operation receiver 31 is implemented by, for example, a touch panel or a hardware button.

When operation receiver 31 has received a predetermined operation, controller 32 causes communicator 33 to transmit a control signal for controlling lighting device 40.

When operation receiver 31 has received a predetermined operation, controller 32 also causes communicator 33 to transmit a request for the health condition information. When, as a result of transmission of this request, communicator 33 has acquired the health condition information transmitted from management device 20, judgment unit 32a of controller 32 judges whether each of users A to D is the first subject or the second subject, on the basis of the acquired health condition information.

Specifically, controller 32 (judgment unit 32a) is implemented by, for example, a processor, a microcomputer, or a dedicated circuit.

Communicator 33 transmits a control signal to lighting device 40 and transmits a request for the health condition information to management device 20 under the control of controller 32. Communicator 33 is one example of an acquisition unit and acquires (receives) the health condition information from management device 20. Specifically, communicator 33 is a communication module (communication circuit) that allows presentation device 30 to communicate with management device 20 and lighting device 40.

The communication between presentation device 30 and management device 20 may be wired communication, or may be wireless communication. There are no particular limitations on communication standards for use in this communication. The communication between presentation device 30 and lighting device 40 may be wired communication, or may be wireless communication. There are no particular limitations on communication standards for use in this communication. When the communication standards used between presentation device 30 and management device 20 differs from the communication standards used between presentation device 30 and lighting device 40, communicator 33 may be configured by a plurality of types of communication modules (communication circuits).

Presentation unit 34 presents the results of judgment made by judgment unit 32a. That is, presentation unit 34 presents whether each of users A to D is the first subject or the second subject. Specifically, presentation unit 34 may be a display that displays whether each of users A to D is the first subject or the second subject, and displays the image as illustrated in FIG. 3.

Storage 35 is a storage device that stores, for example, control programs used by controller 32. Specifically, storage 35 is implemented by, for example, a semiconductor memory.

Lighting Device

Lighting device 40 is a device that illuminates space 50 and that emits the first light and the second light for phototherapy. Lighting device 40 includes communicator 41, irradiation controller 42, light source unit 43, timer 44, and storage 45.

Communicator 41 receives (acquires) the control signal transmitted from presentation device 30 through wireless communication. Specifically, communicator 41 is a communication module (communication circuit) that allows lighting device 40 to communicate with presentation device 30. The communication between lighting device 40 and presentation device 30 may be wired communication, or may be wireless communication. There are no particular limitations on communication standards for use in this communication.

According to Embodiment 1, lighting device 40 operates in accordance with an operation made to presentation device 30. That is, lighting device 40 operates on the basis of the control signal received by communicator 41. Alternatively, lighting device 40 may include a dedicated operation panel and operate independently of presentation device 30. In this case, communicator 41 may be omitted.

When communicator 41 has received a control signal, irradiation controller 42 causes light source unit 43 to emit the first light according to the first irradiation schedule so as to form first area 51 that is irradiated with the first light in space 50. Simultaneously with the formation of first area 51, irradiation controller 42 also causes light source unit 43 to emit the second light according to the second irradiation schedule different from the first irradiation schedule so as to form second area 52 that is irradiated with the second light in space 50. In the example illustrated in FIG. 1, irradiation controller 42 causes first light source 43a and second light source 43b of light source unit 43 to emit the first light according to the first irradiation schedule so as to form first area 51 in space 50. Irradiation controller 42 also causes third light source 43c and fourth light source 43d of light source unit 43 to emit the second light according to the second irradiation schedule so as to form second area 52 in space 50.

Specifically, irradiation controller 42 is implemented by, for example, a processor, a microcomputer, or a dedicated circuit. The first and second irradiation schedules may be information that indicates a correspondence between time and a value of light intensity (brightness) of light source unit 43 at the time, and may be stored in advance in storage 45. Irradiation controller 42 reads out and uses the first and second irradiation schedules stored in storage 45.

Light source unit 43 is a light source controlled by irradiation controller 42. In the example illustrated in FIGS. 1 and 2, light source unit 43 includes first light source 43a, second light source 43b, third light source 43c, and fourth light source 43d. There are no particular limitations on the specific configuration of light source unit 43 such as the number of light sources included in light source unit 43, and any configuration may be used as long as light source unit 43 is capable of forming first and second areas 51 and 52 at different positions in space 50 by emitting the first light and the second light simultaneously.

First light source 43a, second light source 43b, third light source 43c, and fourth light source 43d are ceiling lights that are mounted on the ceiling that defines space 50 and illuminates space 50. First light source 43a, second light source 43b, third light source 43c, and fourth light source 43d may be ceiling lights that are circular in plan view, or alternatively may be long ceiling lights, downlights, or spotlights.

First light source 43a, second light source 43b, third light source 43c, and fourth light source 43d each emit white light. Each of first light source 43a, second light source 43b, third light source 43c, and fourth light source 43d is a light source that supports at least lighting control (adjustment of illuminance (brightness)). Each of first light source 43a, second light source 43b, third light source 43c, and fourth light source 43d may support toning (adjustment of color temperature).

Alternatively, each of first light source 43a, second light source 43b, third light source 43c, and fourth light source 43d may be capable of emitting monochromatic light instead of, or in addition to, white light. For example, phototherapy that causes light source unit 43 to emit blue monochromatic light is conceivable.

Timer 44 is a timer device that measures the current time, and notifies irradiation controller 42 of the measured time. Timer 44 is used by irradiation controller 42 to cause light source unit 43 to emit light according to irradiation schedules. Specifically, timer 44 may be a real-time clock IC, or may be in any form.

Storage 45 is a storage device that stores information such as the first and second irradiation schedules. Specifically, storage 45 is implemented by, for example, a semiconductor memory.

Operations

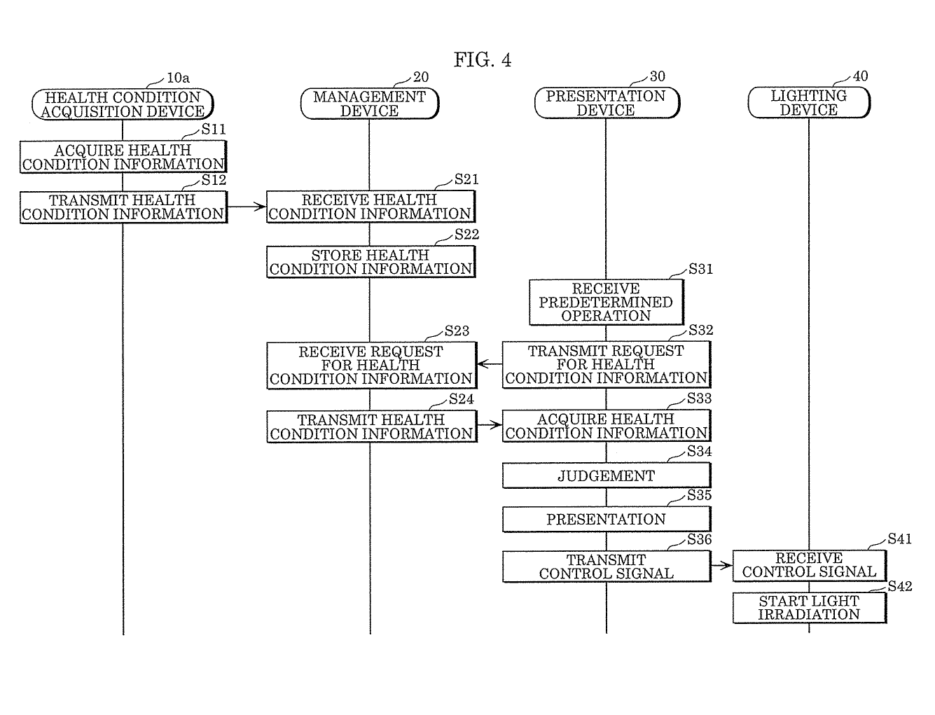

Next, operations of lighting system 100 will be described. FIG. 4 is a sequence diagram illustrating an example of operations of lighting system 100. In FIG. 4, health condition acquisition devices 10b to 10d are not shown in order to simplify the description.

First, health condition acquisition device 10a acquires the health condition information. Health condition acquisition unit 11 of health condition acquisition device 10a acquires the health condition information indicating the health condition of user A (S11), and communicator 12 transmits the health condition information to management device 20 (S12). Although not shown in FIG. 4, the health condition information is also transmitted from health condition acquisition devices 10b to 10d.

Communicator 21 of management device 20 receives the health condition information transmitted from communicator 12 (521). Although not shown, communicator 21 also receives the health condition information from health condition acquisition devices 10b to 10d. Controller 22 stores the health condition information received by communicator 12 (S22).

Meanwhile, when operation receiver 31 of presentation device 30 has received a predetermined operation (operation of starting phototherapy) from staff S (S31), communicator 33 transmits a request for the health condition information under the control of controller 32 (S32). When communicator 21 of management device 20 has received the request for the health condition information (S23), communicator 21 transmits the health condition information under the control of controller 22. At this time, communicator 21 transmits, for example, the health condition information for four users A to D (S24).

When communicator 33 of presentation device 30 has acquired the health condition information (S33), judgment unit 32a performs judgment processing (S34). Specifically, judgment unit 32a judges whether each of users A to D is the first subject or the second subject, on the basis of the acquired health condition information. For example, judgment unit 32a judges users A and B as first subjects and users C and D as second subjects.

Presentation unit 34 performs presentation processing for presenting the results of judgment made by judgment unit 32a (S35). Specifically, presentation unit 34 presents whether each of users A to D is the first subject or the second subject. More specifically, presentation unit 34 presents the image as illustrated in FIG. 3.

Communicator 33 transmits a control signal for controlling lighting device 40 under the control of controller 32 (S36).

When communicator 41 of lighting device 40 has received the control signal (S41), irradiation controller 42 starts photoirradiation based on the first and second irradiation schedules (S42). Specifically, irradiation controller 42 references the first irradiation schedule stored in storage 45 and identifies the intensity of light to be emitted at the current time notified by timer 44. Irradiation controller 42 causes first light source 43a and second light source 43b to emit the first light with the identified light intensity.

Similarly, irradiation controller 42 references the second irradiation schedule stored in storage 45 and identifies the intensity of light to be emitted at the current time notified by timer 44. Irradiation controller 42 causes third light source 43c and fourth light source 43d to emit the second light with the identified light intensity.

In lighting system 100 that performs operations as described above, the users can receive phototherapy according to their health conditions if staff S guides each user to either first area 51 or second area 52 in accordance with the presentation given by presentation unit 34. That is, lighting system 100 can offer phototherapy according to the health conditions of users.

### Specific Example 1

Next, a more specific example of the operations of lighting system 100 will be described. Specific Example 1 describes a case where the health condition information indicates the sleep state of a user.

First, health condition acquisition device 10a will be described. When the health condition information indicates the sleep state of a user, health condition acquisition device 10a (health condition acquisition unit 11) may be a sleep measuring meter that includes a pad-type (sheet-type) sensor as described above. The sleep measuring meter detects whether user A is present on the sensor and detects (acquires) a period of time during which user A is present on the sensor, i.e., a period of time during which user A is sleeping.

Health condition acquisition unit 11 may be an image sensor and an image processor such as a camera. In this case, as a result of the image sensor capturing an image on the bed and the image processor performing image processing on the image, health condition acquisition unit 11 detects (acquires) the period of time during which user A is determined to be present on the bed as the period of time during which user A is sleeping.

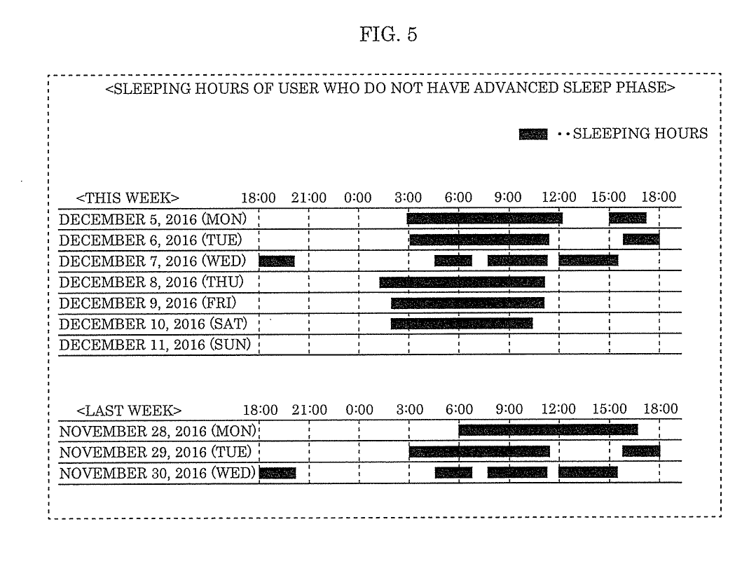

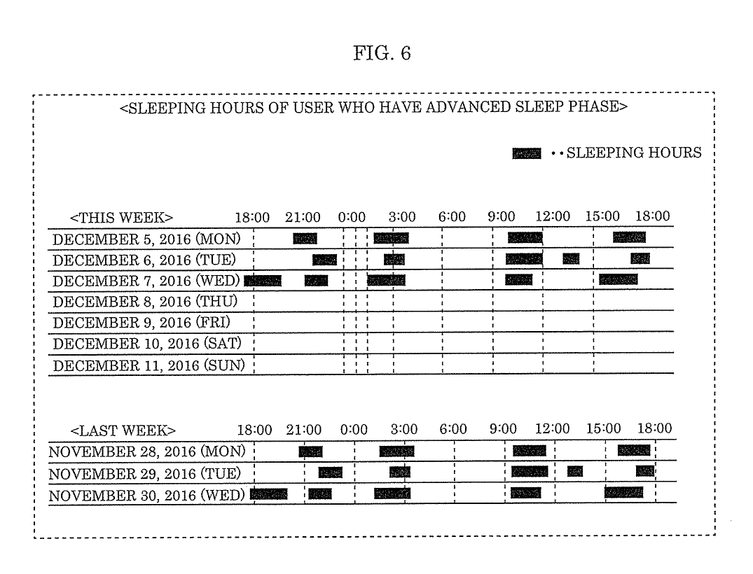

Next, judgment criteria of judgment unit 32a will be described. When the health condition information indicates the sleep state of a user, communicator 33 acquires the health condition information that indicates the sleep states of users A to D in step S33 in FIG. 4. In step S34 in FIG. 4, on the basis of the acquired health condition information, judgment unit 32a judges whether each of users A to D is a first subject who does not have an advanced sleep phase pattern or a second subject who has an advanced sleep phase pattern as compared with the first subject. FIG. 5 illustrates sleeping hours of a first subject who does not have an advanced sleep phase pattern, and FIG. 6 illustrates sleeping hours of a second subject who has an advanced sleep phase pattern.

As illustrated in FIGS. 5 and 6, the first subject who does not have an advanced sleep phase pattern has relatively many sleeping hours that belong to the nighttime (from sunset to sunrise) among the total sleeping hours. On the other hand, the second subject who has an advanced sleep phase pattern has relatively many sleeping hours that belong to the daytime (from sunrise to sunset) among the total sleeping hours.

In view of this, when the user's sleeping hours that belong to the nighttime among the total sleeping hours is greater than or equal to a predetermined ratio according to the health condition information indicating the sleep state of the user, judgment unit 32a judges the user as the first subject who does not have an advanced sleep phase pattern. When the user's sleeping hours that belong the nighttime among the total sleeping time is less than the predetermined ratio according to the health condition information indicating the sleep state of the user, judgment unit 32a judges the user as the second subject who has an advanced sleep phase pattern.

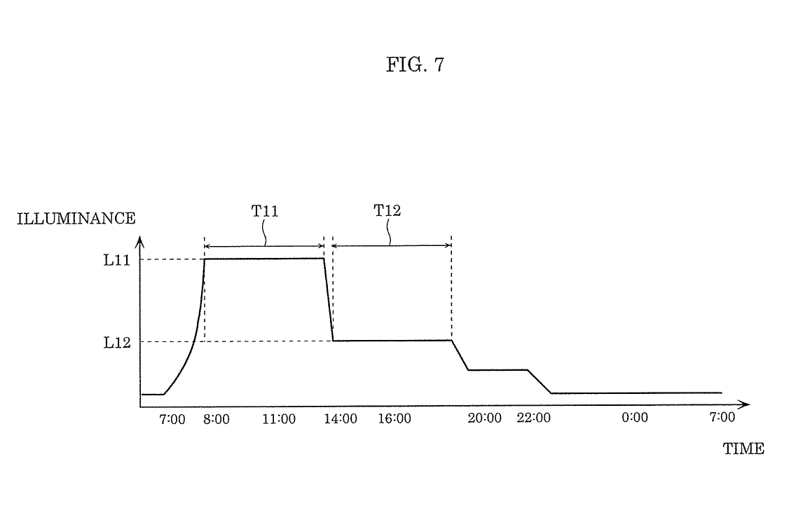

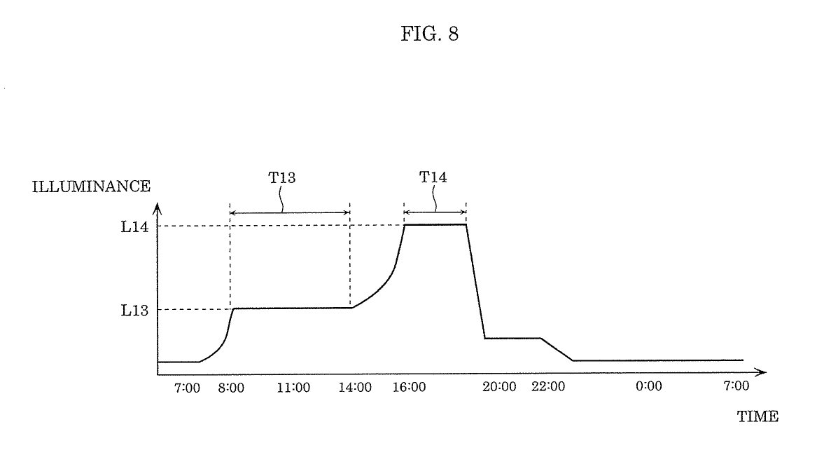

Next, the first and second irradiation schedules that are used when the health condition information indicates the sleep state of a user will be described. FIG. 7 illustrates an example of the first irradiation schedule, and FIG. 8 illustrates an example of the second irradiation schedule. In FIG. 7, the vertical axis represents illuminance at a predetermined position in first area 51 (e.g., desktop surface of the first table). In FIG. 8, the vertical axis represents illuminance at a predetermined position in second area 52 (e.g., desktop surface of the second table). In FIGS. 7 and 8, the horizontal axis represents time.

The first irradiation schedule illustrated in FIG. 7 is an irradiation schedule that makes the illuminance in first area 51 during first period T11 brighter than that during second period T12. First period T11 may be a period of time from around 8:00 to 14:00, and at least part of first period T11 belongs to the morning. Second period T12 may be a period of time from around 14:00 to around 19:00, and entire period T12 belongs to the afternoon. Illuminance L11 during first period T11 is approximately two times illuminance L12 during second period T12.

The first subjects do not have advanced sleep phase patterns. Thus, the first subjects can spend time comfortably while keeping their rhythms of daily life by receiving brighter light in the morning than in the afternoon according to the first irradiation schedule.

On the other hand, the second irradiation schedule illustrated in FIG. 8 is an irradiation schedule that makes the illuminance in second area 52 during third period T13 darker than that during fourth period T14. Third period T13 may be a period of time from around 8:00 to around 14:00, and at least part of third period T13 belongs to the morning. Fourth period T14 is a period of time from around 16:00 to around 19:00, and entire period T14 belongs to the relatively early afternoon. Illuminance L13 during third period T13 is approximately half of illuminance L14 during fourth period T14.

The second subjects have advanced sleep phase patterns. Thus, the second subjects can approximate their sleep phases to a normal condition (condition that is not an advanced sleep phase pattern) by receiving bright light in the early afternoon according to the second irradiation schedule. That is, the rhythms of daily life of the second subjects can be improved.

When light source unit 43 supports toning, the luminous color may be changed depending on the first and second irradiation schedules.

For example, the first irradiation schedule may be an irradiation schedule that emits the first light of a first color during first period T11 and emits the first light of a second color during second period T12, the second color having fewer blue color components than the first color. The blue color components as used herein refer to components with wavelengths of 460 nm to 490 nm.

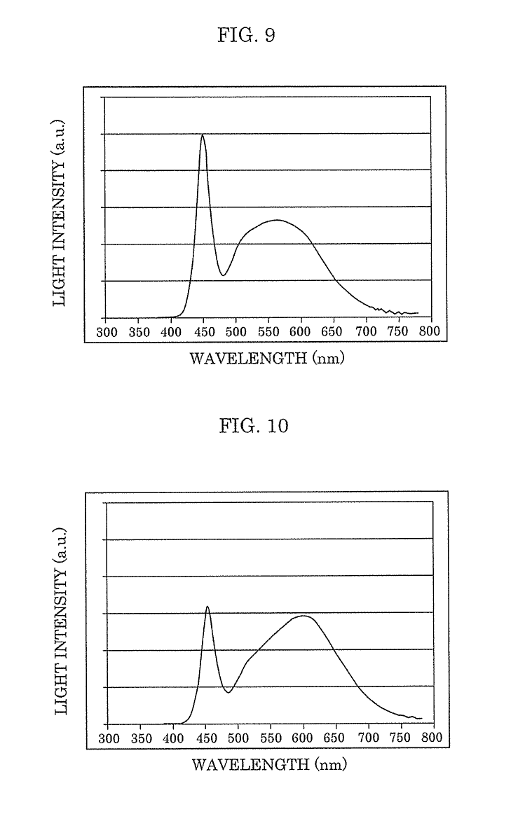

Specifically, the first color may be a first color temperature, and the second color may be a second color temperature. FIG. 9 illustrates an example of an emission spectrum of light with the first color temperature, and FIG. 10 illustrates an example of an emission spectrum of light with the second color temperature.

In this way, by emitting light with more blue color components during the period in which relatively bright light is emitted according to the first irradiation schedule, the effect of phototherapy can be enhanced.

Similarly, the second irradiation schedule may be an irradiation schedule that emits the second light with the second color temperature (second color) during third period T13 and emits the second light with the first color temperature (first color) during fourth period T14.

In this way, by emitting light with more blue color components during the period in which relatively bright light is emitted according to the second irradiation schedule, the effect of the phototherapy can be enhanced.

### Specific Example 2

Specific Example 2 describes a case where the health condition information indicates the degree of dementia of a user.

First, health condition acquisition device 10a will be described. When the health condition information indicates the degree of dementia of users A to D, health condition acquisition device 10a may be a smartphone, a tablet terminal, or a personal computer. For example, each of users A to D takes a dementia test such as Mini-Mental State Examination (MMSE) or Hasegawa Dementia Scale-Revised (HDS-R) and inputs the test result (score) to health condition acquisition devices 10a. In this case, health condition acquisition unit 11 serves as a user interface such as a touch panel or a keyboard. Alternatively, such a dementia test may be conducted directly via health condition acquisition device 10a (information terminal).

Next, judgment criteria of judgment unit 32a will be described. When the health condition information indicates the degree of dementia of a user, communicator 33 acquires the health condition information indicating the degrees of dementia of users A to D in step S33 in FIG. 4. In step S34 in FIG. 4, on the basis of the acquired health condition information, judgment unit 32a judges whether each of users A to D is a first subject with mild dementia or a second subject with more severe dementia than the first subject. Judgement unit 32a uses, for example, a threshold value to judge whether each user is the first subject or the second subject. When a higher dementia test score indicates a more severe degree of dementia, judgment unit 32a judges a user whose dementia test score is less than the threshold value as the first subject, and judges a user whose dementia test score is higher than or equal to the threshold value as the second subject.

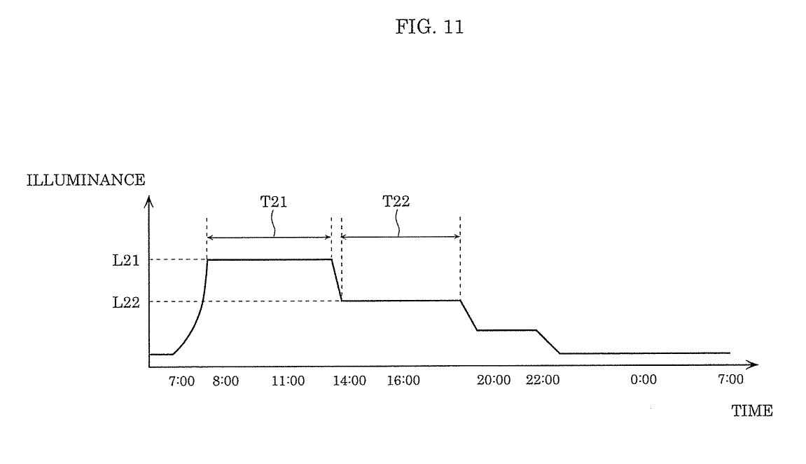

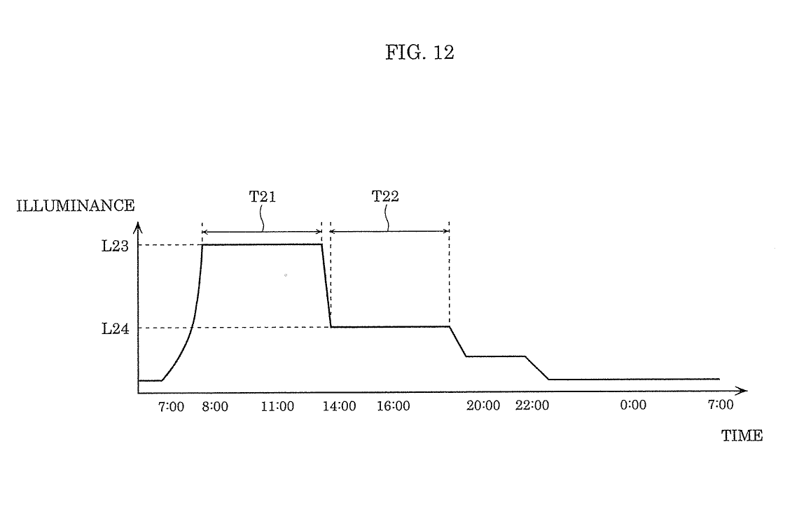

Next, the first and second irradiation schedules that are used when the health condition information indicates the degree of dementia of a user will be described. FIG. 11 illustrates an example of the first irradiation schedule, and FIG. 12 illustrates an example of the second irradiation schedule. In FIG. 11, the vertical axis represents illuminance at a predetermined position in first area 51 (e.g., desktop surface of the first table). In FIG. 12, the vertical axis represents illuminance at a predetermined position in second area 52 (e.g., desktop surface of the second table). In FIGS. 11 and 12, the horizontal axis represents time.

The first irradiation schedule illustrated in FIG. 11 is an irradiation schedule that makes the illuminance in first area 51 during first period T21 brighter than that during second period T22. First period T21 may be a period of time from around 8:00 to around 14:00, and at least part of first period T21 belongs to the morning. Second period T22 is a period of time from around 14:00 to around 19:00, and entire period T22 belongs to the afternoon. Illuminance L21 during first period T21 is in the range of approximately 1.6 to 1.7 times illuminance L22 during second period T22.

On the other hand, the second irradiation schedule illustrated in FIG. 12 is an irradiation schedule that makes the illuminance in second area 52 during first period T21 brighter than that during second period T22. Illuminance L23 during first period T21 is approximately two times or more illuminance L24 during second period T22.

A comparison between the first and second irradiation schedules indicates that the first irradiation schedule provides lower accumulated illuminance during a predetermined period (e.g., a total of the first and second periods) than the second irradiation schedule.

According to the findings of the inventors, the effect of the phototherapy can be enhanced by applying an irradiation schedule that provides accumulated illuminance equivalent to or higher than that provided by common phototherapy to the second subjects with severe dementia. Thus, the first and second irradiation schedules as described above can help offer phototherapy according to the degree of dementia of each user.

When light source unit 43 supports toning, the color of the first light and the color of the second light may differ from each other. For example, the color of the first light may have fewer blue color components than the color of the second light. In this case, specifically speaking, the first light may be light with the aforementioned second color temperature, and the second light may be light with the aforementioned first color temperature.

The fact that the color of the first light has fewer blue color components than the color of the second light brings about an effect similar to that in the case where the first irradiation schedule provides lower accumulated illuminance than the second irradiation schedule. Thus, using the color of the first light with fewer blue color components than the color of the second light can also help offer phototherapy according to the degree of dementia of each user.

When the color of the first light has fewer blue color components than the color of the second light, it is not an absolute necessity for the first irradiation schedule to provide lower accumulated illuminance than the second irradiation schedule, and the first and second irradiation schedules may provide the same accumulated illuminance and may show the same change in illuminance over time.

### Specific Example 3

Specific Example 3 describes a case where the health condition information indicates the activity level of a user.

First, health condition acquisition device 10a will be described. When the health condition information indicates the activity level of a user, health condition acquisition device 10a (health condition acquisition unit 11) may be an activity-level measuring meter attached on the user's body, or may be a mobile terminal such as a smartphone that has a function equivalent to that of the activity-level measuring meter.

Next, judgment criteria of judgment unit 32a will be described. When the health condition information indicates the activity level of a user, communicator 33 acquires the health condition information indicating the activity levels of users A to D in step S33 in FIG. 4. In step S34 in FIG. 4, on the basis of the acquired health condition information, judgment unit 32a judges whether each of users A to D is a first subject with a high activity level or a second subject with an activity level lower than that of the first subject. Judgement unit 32a uses, for example, a threshold value to judge whether each user is the first subject or the second subject. Judgement unit 32a judges a user whose activity level is higher than or equal to the threshold value as the first subject, and judges a user whose activity level is lower than the threshold value as the second subject.

The first and second irradiation schedules that are used when the health condition information indicates the activity level of s user are similar to those used when the health condition information indicates the degree of dementia of a user. A comparison between the first and second irradiation schedules indicates that the first irradiation schedule provides lower accumulated illuminance during a predetermined period (e.g., a total of the first and second periods) than the second irradiation schedule.

According to the findings of the inventors, the effect of the phototherapy can be enhanced by applying an irradiation schedule that provides accumulated illuminance equivalent to or higher than that provided by common phototherapy to the second subjects with low activity levels. Thus, the first and second irradiation schedules as described above can help offer phototherapy according to the activity level of each user.

As described above, the fact that the color of the first light has fewer blue color components than the color of the second light brings about an effect similar to that in the case where the first irradiation schedule provides lower accumulated illuminance than the second irradiation schedule. Thus, even when the health condition information indicates the activity level of a user, using the color of the first light with fewer blue color components than the color of the second light can help offer phototherapy according to the activity level of each user.

### Variation 1

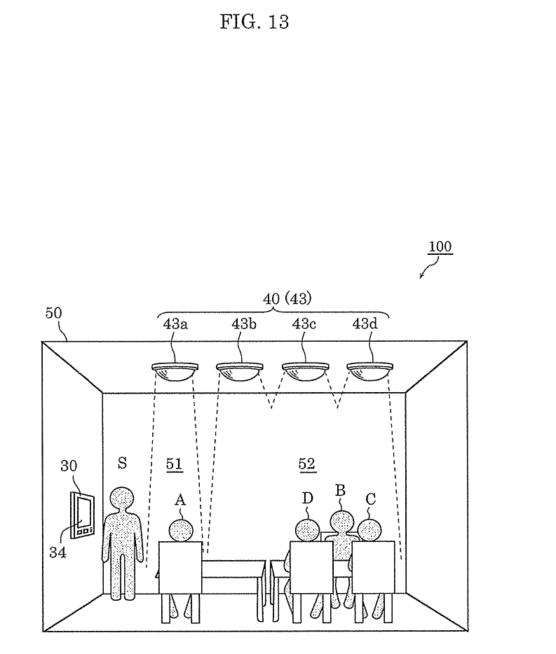

According to Embodiment 1 described above, first area 51 and second area 52 are of approximately the same size as illustrated in FIG. 1. Alternatively, irradiation controller 42 may change the sizes of first area 51 and second area 52 according to the number of users judged as the first subjects by judgment unit 32a and the number of users judged as the second subjects by judgment unit 32a. FIG. 13 is a diagram for illustrating how irradiation controller 42 changes the sizes of first area 51 and second area 52.

For example, irradiation controller 42 may acquire information indicating the number of first subjects and the number of second subjects from judgment unit 32a and change the sizes of first area 51 and second area according to the ratio of the number of first subjects to the number of second subjects. Specifically, irradiation controller 42 controls the number of light sources that emit the first light and the number of light sources that emit the second light.

For example, when the number of first subjects and the number of second subjects are approximately equal, irradiation controller 42 causes the two light sources, namely, first light source 43a and second light source 43b to emit the first light and causes the two light sources, namely, third light source 43c and fourth light source 43d to emit the second light as illustrated in FIG. 1. That is, irradiation controller 42 equalizes the number of light sources that emit the first light and the number of light sources that emit the second light. Thus, first area 51 and second area 52 become approximately the same size.

On the other hand, when the number of first subjects is smaller than the number of second subjects, irradiation controller 42 causes first light source 43a to emit the first light and causes the three light sources, namely, second light source 43b, third light source 43c, and fourth light source 43d to emit the second light as illustrated in FIG. 13. That is, irradiation controller 42 reduces the number of light sources that emit the first light to be smaller than the number of light sources that emit the second light. As a result, the size of first area 51 becomes smaller than the size of second area 52. In the example illustrated in FIG. 13, the ratio between the number of first subjects and the number of second subjects, i.e., the ratio between the size (area) of first area 51 and the size (area) of second area 52, is 1:3.

Under the control of irradiation controller 42 as described above, the sizes of first area 51 and second area 52 can be appropriately changed according to the number of first subjects and the number of second subjects.

### Variation 2

While irradiation controller 42 forms the two areas, namely, first area 51 and second area 52 in single space 50 according to Embodiment 1 described above, irradiation controller 42 may form three or more irradiation areas using different irradiation schedules.

In this case, judgment unit 32a judges which one of three or more attributes each user belongs to according to his or her health condition (i.e., whether the user is a first subject, a second subject, or a third subject, and so on). For example, judgment unit 32a judges which of three or more attributes (e.g., three attributes of the degree of dementia, i.e., mild, medium, and severe) each user belongs to according to one of the following: sleep state, degree of dementia, and activity level.

Alternatively, judgment unit 32a may use two of the following: sleep state, degree of dementia, and activity level to judge which of three or more attributes each user belongs to. As another alternative, for example, judgment unit 32a may judge which of four attributes (delayed sleep phase and mild dementia, delayed sleep phase and severe dementia, advanced sleep phase and mild dementia, and advanced sleep phase and severe dementia) each user belongs to, by judging whether the user has an advanced sleep phase pattern and whether the degree of dementia of the user is severe.

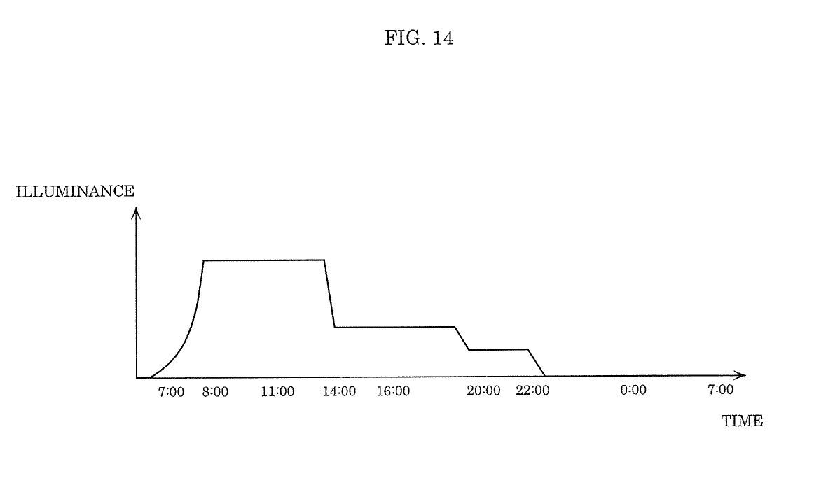

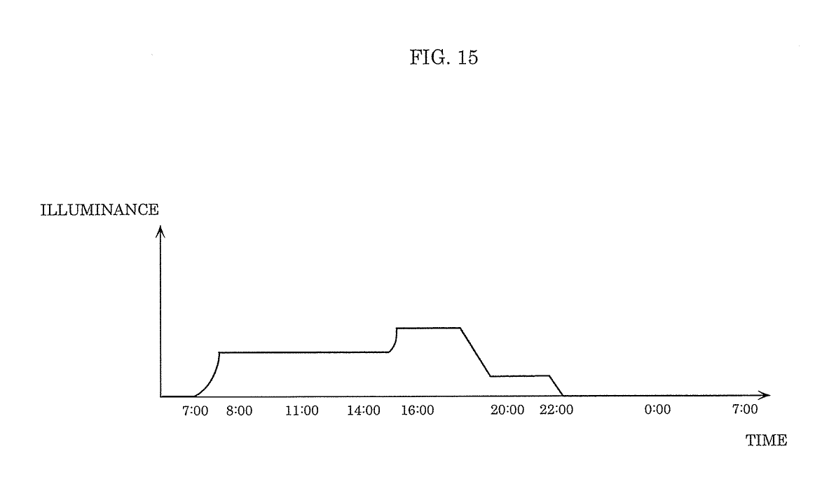

An irradiation schedule used for users who are judged to have a delayed sleep phase and severe dementia is as illustrated in FIG. 14, and an irradiation schedule used for users who are judged to have an advanced sleep phase and mild dementia are as illustrated in FIG. 15. FIG. 14 illustrates an irradiation schedule for users who are judged to have a delayed sleep phase and severe dementia, and FIG. 15 illustrates an irradiation schedule for users who are judged to have an advanced sleep phase and mild dementia.

As described above, by forming three or more areas using different irradiation schedules in single space 50, lighting system 100 can offer phototherapy that meticulously responds to the detail of the health conditions of users.

### Variation 3

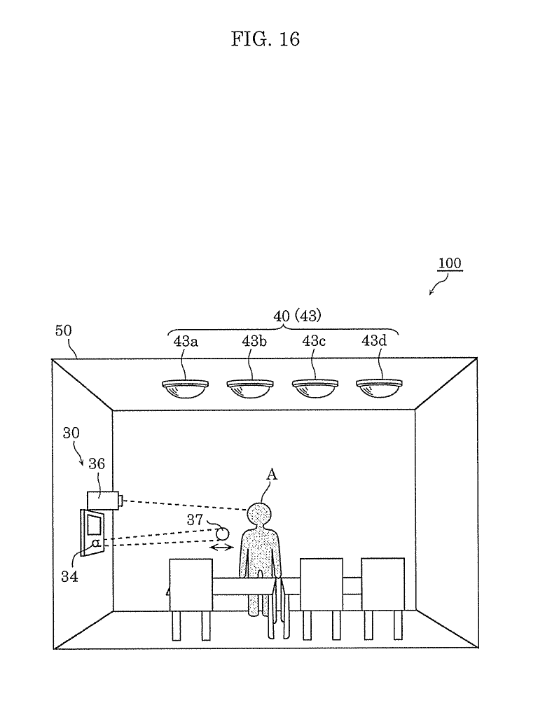

While presentation device 30 presents whether each of users A to D is the first subject or the second subject on the display (presentation unit 34) according to Embodiment 1 described above, there are no particular limitations on a specific mode of presentation unit 34. For example, presentation device 30 may employ light projection to present whether each of users A to D is the first subject or the second subject. In this case, presentation unit 34 may be a light source unit capable of projecting light. Alternatively, presentation device 30 may present information directly to users A to D, instead of staff S. FIG. 16 illustrates an example of a variation of the way of presentation by presentation device 30.

In the example illustrated in FIG. 16, presentation device 30 further includes image sensor 36 that captures an image including the face of user A who has entered space 50. Controller 32 is capable of identifying the user who has entered space 50 through image processing (facial recognition processing) performed on the image captured by image sensor 36. In this case, a database that associates IDs of a plurality of users with facial recognition patterns corresponding to the faces of the users is stored in advance in storage 35, and controller 32 is capable of identifying user A who has entered space 50 by checking the recognition pattern obtained from the captured image against the facial recognition patterns stored in storage 35.

In FIG. 16, presentation unit 34 serves as a light projector that projects light and is capable of forming beam spot 37 in the vicinity of user A who has entered space 50. Judgement unit 32a judges whether the identified user A is the first subject or the second subject, and when user A is judged as the first subject, presentation unit 34 moves beam spot 37 to first area 51. When user A is judged as the second subject, presentation unit 34 moves beam spot 37 to second area 52. In this way, presentation device 30 can guide user A to either first area 51 or second area 52. Alternatively, staff S may guide user A on the basis of this presentation.

### Effects of Embodiment 1

Lighting system 100 includes light source unit 43, irradiation controller 42, communicator 33, judgment unit 32a, and presentation unit 34. Irradiation controller 42 forms first area 51 that is irradiated with the first light in space 50 by causing light source unit 43 to emit the first light according to the first irradiation schedule, and simultaneously with the formation of first area 51, forms second area 52 that is irradiated with the second light in space 50 by causing light source unit 43 to emit the second light according to the second irradiation schedule different from the first irradiation schedule. Communicator 33 acquires the health condition information indicating the health conditions of users. Judgement unit 32a judges whether each user is the first subject targeted for irradiation with the first light or the second subject targeted for irradiation with the second light, on the basis of the acquired health condition information. Presentation unit 34 presents the results of the above judgment. Communicator 33 is one example of the acquisition unit.

Thus, users can receive phototherapy according to their health conditions if staff S guides the users to either first area 51 or second area 52 on the basis of the presentation given by presentation unit 34. That is, lighting system 100 can offer phototherapy according to the health condition of each user.

Alternatively, judgment unit 32a may judge whether each of a plurality of users is the first subject or the second subject, and irradiation controller 42 may change the sizes of first area 51 and second area 52 according to the number of users who are judged as the first subjects by judgment unit 32a and the number of users who are judged as the second subjects by judgment unit 32a.

As a result, lighting system 100 can appropriately change the sizes of first area 51 and second area 52 according to the number of users who are judged as the first subjects and the number of users who are judged as the second subjects.

Communicator 33 may acquire health condition information that indicates the sleep state of a user. Judgement unit 32a may judge whether each user is a first subject or a second subject who has an advanced sleep phase pattern as compared with the first subject, on the basis of the acquired health condition information. The first irradiation schedule may be an irradiation schedule that makes the illuminance in first area 51 during first period T11 brighter than that during second period T12, at least part of first area 51 belonging to the morning, and the second period belonging to the afternoon. The second irradiation schedule may be an irradiation schedule that makes the illuminance in second area 52 during third period T13 darker than that during fourth period T14, at least part of the third period belonging to the morning, and the fourth period belonging to the afternoon.

In this case, users who are judged as the first subjects can stay comfortably while keeping their rhythms of daily life by receiving brighter light in the morning than in the afternoon. Users who are judged as the second subjects can approximate their sleep phases to the normal condition (condition that is not an advanced sleep phase pattern) by receiving bright light in the early afternoon, and can thereby improve the rhythms of daily life.

The first irradiation schedule may be an irradiation schedule that emits the first light of the first color during first period T11 and emits the first light of the second color during second period T12, the second color having fewer blue color components than the first color. The second irradiation schedule may be an irradiation schedule that emits the second light of the second color during third period T13 and emits the second light of the first color during fourth period T14.

This improves the effect of the phototherapy.

Communicator 33 may acquire the health condition information indicating the degree of dementia of a user. Judgement unit 32a may judge whether each user is a first subject or a second subject with more severe dementia than the first subject, on the basis of the acquired health condition information. The first irradiation schedule may be an irradiation schedule that provides lower accumulated illuminance during a predetermined period than the second irradiation schedule.

As a result, the second irradiation schedule that provides higher accumulated illuminance than the first irradiation schedule is applied to users who are judged as the second subjects with severe dementia. This improves the effect of the phototherapy.

Communicator 33 may acquire the health condition information indicating the activity level of a user, and on the basis of the acquired health condition information, judgment unit 32a may judge whether each user is a first subject or a second subject with an activity level lower than that of the first subject. The first irradiation schedule may be an irradiation schedule that provides lower accumulated illuminance during a predetermined period than the second irradiation schedule.

As a result, the second irradiation schedule that provides higher accumulated illuminance than the first irradiation schedule is applied to users who are judged as the second subjects with low activity levels. This improves the effect of the phototherapy.

The first irradiation schedule may be an irradiation schedule that makes the illuminance in first area 51 during first period T21 brighter than that during second period T22, at least part of first period T21 belonging to the morning, and second period T22 belonging to the afternoon. The second irradiation schedule may be an irradiation schedule that makes the illuminance in second area 52 during first period T21 brighter than that during second period T22.

This improves the effect of the phototherapy.

The color of the first light may have fewer blue color components than the color of the second light.

This also improves the effect of the phototherapy.

Presentation unit 34 may be a display that displays judgment results.

In this case, staff S is able to recognize whether each user is the first subject or the second subject according to the image.

### Embodiment 2

Overview of Lighting System

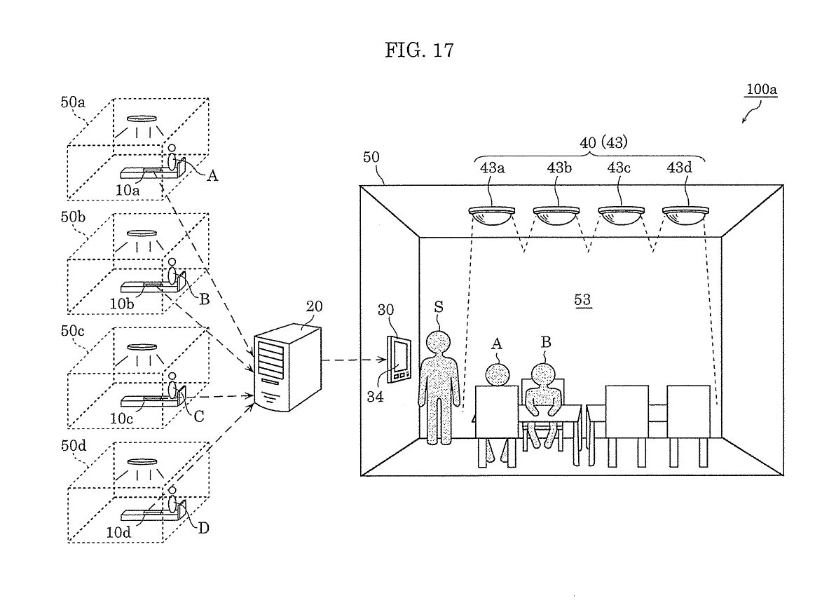

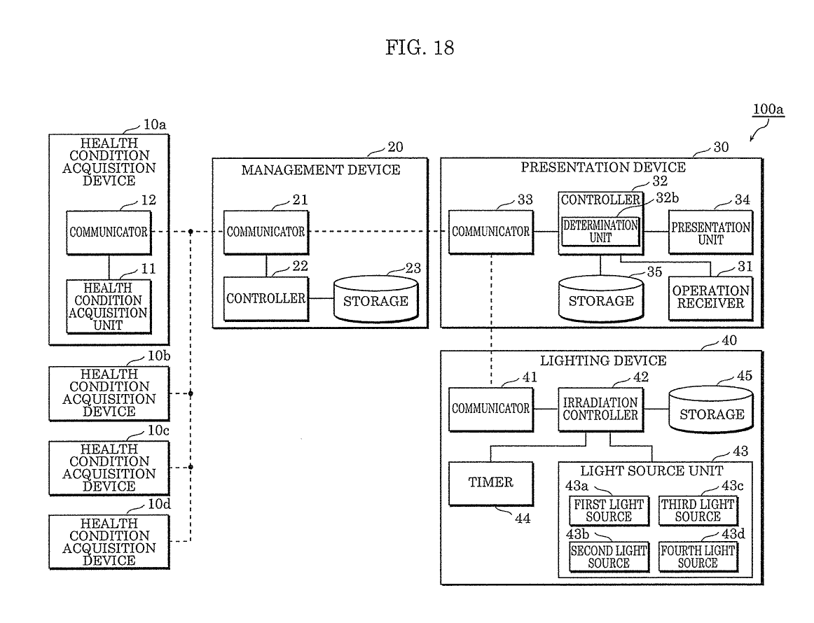

Next, a configuration of a lighting system according to Embodiment 2 will be described. FIG. 17 illustrates an overview of the lighting system according to Embodiment 2. FIG. 18 is a block diagram illustrating a functional configuration of the lighting system according to Embodiment 2. The following description of Embodiment 2 focuses on differences from Embodiment 1, and a redundant description that overlaps with that of Embodiment 1 may be appropriately omitted.

As illustrated in FIG. 17, lighting system 100a according to Embodiment 2 forms single irradiation area 53 in space 50. That is, irradiation controller 42 forms irradiation area 53 that is irradiated with light in space 50 by causing light source unit 43 to emit light according to a first irradiation schedule. The light to be emitted at this time is bright light for use in phototherapy (e.g., light with illuminance L11 in FIG. 7). That is, space 50 is always illuminated by the light for use in phototherapy.

As illustrated in FIG. 18, controller 32 of lighting system 100a according to Embodiment 2 includes determination unit 32b. Determination unit 32b determines periods of time during which users A to D are supposed to stay in irradiation area 53, on the basis of information indicating the sleep state acquired by communicator 33.

Specifically, when user A is a first subject who does not have an advanced sleep phase pattern, determination unit 32b determines a first period as a period of time during which user A is supposed to stay in irradiation area 53, on the basis of the acquired health condition information. The first period may be a period of time that belongs at least in part to the morning, and may be a period of time from around 8:00 to around 14:00. When user A is a second subject who has an advanced sleep phase pattern as compared with the first subject, determination unit 32b determines a second period as a period of time during which user A is supposed to stay in irradiation area 53. The second period may be a period of time that belongs to the afternoon in its entirety, and may be a period of time from around 16:00 to around 19:00.

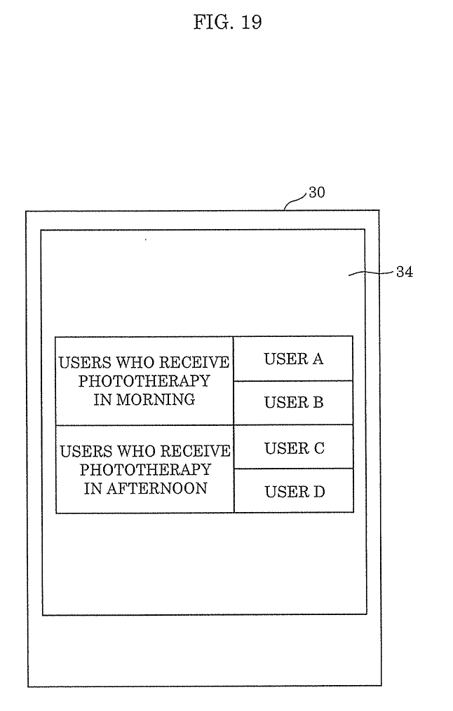

Presentation unit 34 presents the period of time determined by determination unit 32b. FIG. 19 illustrates an example of an image that is displayed by presentation unit 34. FIG. 19 illustrates an image that is displayed when users A and B are first subjects, and users C and D are second subjects. The periods of time presented by presentation unit 34 as illustrated in FIG. 19 allows staff S to guide users A to D to space 50 during the presented periods of time.

As described above, the first subjects who do not have advanced sleep phase patterns can stay comfortably while keeping their rhythms of daily life by receiving brighter light in the morning than in the afternoon. On the other hand, the second subjects who have advanced sleep phase patterns can approximate their sleep phases to the normal condition (condition that is not an advanced sleep phase) by receiving bright light in the early afternoon. That is, the rhythms of daily life of the second subjects can be improved.

Thus, by presenting a period of time suitable for phototherapy as illustrated in FIG. 19, lighting system 100a can offer phototherapy according to the sleep states of users A to D, even if staff S has no expert knowledge.

### Effects of Embodiment 2

As described above, lighting system 100a includes light source unit 43, irradiation controller 42, communicator 33, determination unit 32b, and presentation unit 34. Irradiation controller forms irradiation area 53 that is irradiated with light in space 50 by causing light source unit 43 to emit light. Communicator 33 acquires information indicating the sleep state of a user. Determination unit 32b determines a period of time during which a user is supposed to stay in irradiation area 53, on the basis of the acquired information. Presentation unit 34 presents the period of time determined by determination unit 32b.

Thus, users can receive phototherapy according to their sleep state if the staff S guides the users to irradiation area 53 during the periods of time presented by presentation unit 34. That is, lighting system 100a can offer phototherapy according to the sleep state of each user.

When a user is the first subject, determination unit 32b may determine the first period as the aforementioned period of time on the basis of the acquired information, at least part of the first period belonging to the morning. When a user is the second subject who has an advanced sleep phase pattern as compared with the first subject, determination unit 32b may determine the second period that belongs to the afternoon as the aforementioned period of time.

As a result, users who are judged as the first subjects can stay comfortably while keeping their rhythms of daily life by receiving brighter light in the morning than in the afternoon. Users who are judged as the second subjects can approximate their sleep phases to the normal condition (condition that is not an advanced sleep phase) and improve their rhythms of daily life by receiving bright light in the early afternoon.

### Other Embodiments

While the above has been a description of the lighting systems according to embodiments, the present disclosure is not intended to be limited to the embodiments described above.

For example, there are no particular limitations on the method of communication between devices described in the above embodiments. When wireless communication is carried out between devices, examples of wireless communication systems (communication standards) include specific low-power wireless communication using a frequency of 920 MHz, such as ZigBee (Registered Trademark), Bluetooth (Registered Trademark), and a wireless local area network (LAN). Instead of wireless communication, wired communication may be carried out between devices. Specific examples include power-line carrier communication (PLC) and communication using wire LANs.

In the above-described embodiments, processing executed by specific processors may be implemented by other processors. A sequence of a plurality of processes may be changed, and a plurality of processes may be executed simultaneously.

Dividing the constituent elements of the lighting systems into a plurality of devices is merely one example. For example, the management device may include a judgment unit and a determination unit, and the presentation device may present judgment results acquired from the management device. The lighting systems do not necessarily have to include the management device, and in this case, the presentation device does not necessarily have to acquire the health condition information directly from the health condition acquisition devices.

In the above-described embodiments, constituent elements such as a controller may be configured by dedicated hardware, or may be implemented by executing a software program suitable for each constituent element. Each constituent element may also be implemented by a program executor such as a CPU or a processor reading out and executing a software program stored in a hard disk or a recording medium such as a semiconductor memory.

Constituent elements such as a controller may be circuits (or integrated circuits). These circuits may constitute a single circuit as a unit, or may be separate circuits. These circuits may also be general-purpose circuits or dedicated circuits.

It is to be noted that general or specific embodiments of the present disclosure may be implemented as a system, a method, an integrated circuit, a computer program, or a computer-readable recording medium such as a CD-ROM, or may be implemented as any combination of a system, a method, an integrated circuit, a computer program, or a recording medium. For example, when a general-purpose information terminal such as a smartphone or a tablet terminal is used as the presentation device, the present disclosure may be implemented as an application program for causing the information terminal (computer) to function as the presentation device. The present disclosure may also be implemented as an information presentation method that is executed by a lighting system. The present disclosure may also be implemented as a presentation device that is included in a lighting system.

The present disclosure also includes other variations obtained by applying various changes conceivable by a person skilled in the art to each embodiment, and variations obtained by any combination of the constituent elements and functions of each embodiment without departing from the scope of the present disclosure.

While the foregoing has described one or more embodiments and/or other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present teachings.

What is claimed is:

1. A lighting system comprising:
a light source unit;
an irradiation controller that forms a first area that is irradiated with first light in a space by causing the light source unit to emit the first light according to a first irradiation schedule, and simultaneously with formation of the first area, forms a second area that is irradiated with second light in the space by causing the light source unit to emit the second light according to a second irradiation schedule different from the first irradiation schedule;
an acquisition unit that acquires health condition information that indicates a health condition of a user;
a judgment unit that performs judgment as to whether the user is a first subject targeted for irradiation with the first light or a second subject targeted for irradiation with the second light, in accordance with the health condition information acquired; and
a presentation unit that presents a result of the judgment.

2. The lighting system according to claim 1,
wherein the judgment unit performs the judgment as to whether the user is the first subject or the second subject, for each of a plurality of users, and
the irradiation controller changes sizes of the first area and the second area according to the number of users each judged as the first subject by the judgment unit and the number of users each judged as the second subject by the judgment unit.

3. The lighting system according to claim 1,
wherein the acquisition unit acquires the health condition information that indicates a sleep state of the user,
the judgment unit performs judgment as to whether the user is the first subject or the second subject who has an advanced sleep phase pattern as compared with the first subject, in accordance with the health condition information acquired,
the first irradiation schedule is an irradiation schedule that makes illuminance in the first area during a first period brighter than illuminance in the first area during a second period, at least part of the first period belonging to a morning, and the second period belonging to an afternoon, and
the second irradiation schedule is an irradiation schedule that makes illuminance in the second area during a third period darker than illuminance in the second area during a fourth period, at least part of the third period belonging to the morning, and the fourth period belonging to the afternoon.

4. The lighting system according to claim 3,
wherein the first irradiation schedule is an irradiation schedule for emitting the first light of a first color during the first period and emitting the first light of a second color during the second period, the second color having fewer blue color components than the first color, and
the second irradiation schedule is an irradiation schedule for emitting the second light of the second color during the third period and emitting the second light of the first color during the fourth period.

5. The lighting system according to claim 1,
wherein the acquisition unit acquires the health condition information that indicates a degree of dementia of the user,
the judgment unit performs judgment as to whether the user is the first subject or the second subject with more severe dementia than the first subject, in accordance with the health condition information acquired, and
the first irradiation schedule is an irradiation schedule that provides lower accumulated illuminance during a predetermined period than the second irradiation schedule.

6. The lighting system according to claim 1,
wherein the acquisition unit acquires the health condition information that indicates an activity level of the user,
the judgment unit performs judgment as to whether the user is the first subject or the second subject with an activity level lower than an activity level of the first subject, in accordance with the health condition information acquired, and
the first irradiation schedule is an irradiation schedule that provides lower accumulated illuminance during a predetermined period than the second irradiation schedule.

7. The lighting system according to claim 5,
wherein the first irradiation schedule is an irradiation schedule that makes illuminance in the first area during a first period brighter than illuminance in the first area during a second period, at least part of the first period belonging to a morning, and the second period belonging to an afternoon, and
the second irradiation schedule is an irradiation schedule that makes illuminance in the second area during the first period brighter than illuminance in the second area during the second period.

8. The lighting system according to claim 5,
wherein a color of the first light has fewer blue color components than a color of the second light.

9. The lighting system according to claim 1,
wherein the presentation unit is a display that displays a result of the judgment.

10. A lighting system comprising:
a light source unit;
an irradiation controller that forms an irradiation area that is irradiated with light in a space by causing the light source unit to emit the light;
an acquisition unit that acquires information indicating a sleep state of a user;
a determination unit that determines a period of time during which the user is supposed to stay in the irradiation area, in accordance with the information acquired; and
a presentation unit that presents the period of time determined by the determination unit.

11. The lighting system according to claim 10,
wherein, when the user is a first subject, the determination unit determines a first period as the period of time in accordance with the information acquired, at least part of the first period belonging to a morning, and
when the user is a second subject who has an advanced sleep phase pattern as compared with the first subject, the determination unit determines a second period that belongs to an afternoon as the period of time, in accordance with the information acquired.

12. An information presentation method for carrying out phototherapy in a space that includes a first area and a second area,
the first area being an area that is irradiated with first light according to a first irradiation schedule,
the second area being an area that is irradiated with second light according to a second irradiation schedule different from the first irradiation schedule,
the information presentation method comprising:
acquiring health condition information that indicates a health condition of a user;
performing judgment as to whether the user is a first subject targeted for irradiation with the first light or a second subject targeted for irradiation with the second light, in accordance with the health condition information acquired; and
presenting a result of the judgment.

13. A non-transitory computer-readable recording medium for use in a computer, the recording medium having a computer program recorded thereon for causing the computer to execute the information presentation method according to claim 12.

14. An information presentation method for carrying out phototherapy in a space that includes an irradiation area that is irradiated with light by a light source unit,
the information presentation method comprising:
acquiring information that indicates a sleep state of a user;
determining a period of time during which the user is supposed to stay in the irradiation area, in accordance with the information acquired; and
presenting the determined period of time.

15. A non-transitory computer-readable recording medium for use in a computer, the recording medium having a computer program recorded thereon for causing the computer to execute the information presentation method according to claim 14.
